(19) United States
(12) Patent Application Publication
(10) Pub. No.: US 2023/0093063 A1
(43) Pub. Date: Mar. 23, 2023

(54) DEVICES INCLUDING FERROELECTRIC NEMATIC MATERIAL AND METHODS OF FORMING AND USING SAME

(71) Applicants: The Regents of the University of Colorado, a body corporate, Denver, CO (US); University of Utah Research Foundation, Salt Lake City, UT (US)

(72) Inventors: Noel A. Clark, Boulder, CO (US); Xi Chen, Boulder, CO (US); Matthew A. Glaser, Boulder, CO (US); Joseph E. Maclennan, Boulder, CO (US); Dengpan Dong, Salt Lake City, UT (US); Dimitry Bedrov, Sandy, UT (US)

(21) Appl. No.: 17/909,276

(22) PCT Filed: Mar. 3, 2021
(86) PCT No.: PCT/US2021/020741

§ 371 Date: Sep. 2, 2022

Related U.S. Application Data
(60) Provisional application No. 62/984,739, filed on Mar. 3, 2020.

(51) Int. Cl. G02F 1/141; G02F 1/135; G02F 1/01; G02F 1/00; G21K 1/00; G16C 10/00; G16C 20/20

(52) U.S. Cl. CPC G02F 1/141; G02F 1/1358; G02F 1/0136; G02F 1/0045; G21K 1/00; G16C 10/00; G16C 20/20; G02F 2202/01

(57) ABSTRACT

Devices including nematic liquid crystal-forming molecules are disclosed. The molecules include one or more dipoles and exist in a ferroelectric nematic state. Exemplary devices can further include an electrode for applying an electric field in, for example, and in-plane direction.

### CROSS-REFERENCE TO RELATED APPLICATIONS

This application claims the benefit of U.S. Provisional Application Ser. No. 62/984,739, filed Mar. 3, 2020, entitled DEVICES INCLUDING FERROELECTRIC NEMATIC MATERIAL AND METHODS OF FORMING AND USING SAME, the contents of which are hereby incorporated herein by reference, to the extent such contents do not conflict with the present disclosure.

### STATEMENT REGARDING FEDERALLY-SPONSORED RESEARCH OR DEVELOPMENT

This invention was made with government support under grant number DMR1420736 awarded by the National Science Foundation. The government has certain rights in the invention

### BACKGROUND OF THE DISCLOSURE

Nematic liquid crystals are materials of anisotropically shaped molecules or particles, which, when packed together in a condensed phase, achieve a uniform mutual orientation. For example, rod shaped molecules orient with their long axes tending to be locally aligned along a common direction. This orientational ordering has the beneficial effects of making the material optically anisotropic (birefringent) and of enhancing a response to the application of external influences, such as electric or magnetic fields. Such responsive liquid crystals are widely useful as the electro-optical elements that makes mobile phone display, computer monitor, TV display technology, and the like possible. Display applications employ nematic liquid crystals, which are liquid, but in general, nematic liquid crystals can be liquid, viscoelastic, or glassy, and made of molecular species that are monomeric, oligomeric, or polymeric. For purposes of this disclosure we will refer to these various partially fluid-like, partially solid-like liquid crystal materials types as “nematic” and “fluid.”

In addition to their steric rod shape (e.g., like a hot dog) molecules making nematic liquid crystal phases may be polar, with one end differing from the other (e.g., like a baseball bat or an arrow). Molecular polarity can be introduced by, for example, adopting internal molecular structure that is “dipolar,” in which the internal electrical charge distribution inside the molecule is not spatially uniform, but rather has separated regions of excess positive or negative charge (dipoles). Molecules with dipoles have the possibility of the additional kind of ordering in which the molecular arrows come to point in the same direction (polar ordering). For example, rod-shaped molecules with the dipole arrow along their long axis can spontaneously order parallel and with the dipoles all in the same direction, like the arrows in a quiver or those stuck in a target. If such ordering occurs in a nematic liquid crystal, then resulting material can be said to be optimally “ferroelectric.”

Ferroelectric fluids are interesting because, according to recent modeling, having an optimally common orientation of the dipoles ought to make the response of the fluid to applied electric field much greater than that of a fluid without the polar ordering, for example, molecules should change their orientation in response to applied voltage at much lower voltages. However, heretofore, such beneficial effects have never been observed. One difficulty to be overcome is having to achieve sufficiently large volumes (domains) of material having polar order. For example, some nematic materials may achieve polar ordering in arrays of columns or slabs of material, but where neighboring columns or slabs in the array order with the polarization in the opposite direction, cancelling the overall polarity within a functional volume. Such ordering is referred to as being “antiferroelectric,” and offers little advantage in enhancing the electrical response of the fluid. A ferroelectric nematic fluid will expel antiferroelectric domains.

A ferroelectric nematic liquid crystal will also exhibit preferred orientations at surfaces as is known in the art of nonferroelectric nematic liquid crystals, and in addition, since surfaces are unavoidably polar, will exhibit polar interaction of the ferroelectric polarization with surfaces. These polar and nonpolar surface interactions can be used to obtain desired geometrical organization of the ferroelectric nematic molecular orientation field. Thus, the defining characteristic of a ferroelectric nematic is that it can achieve fixed patterns of polar order, limited only by unavoidable thermal fluctuations, over macroscopic volumes ranging from the nanometer scale and larger, stabilized only by its interaction with the bounding surfaces.

Improved devices including ferroelectric nematic fluids, particularly devices without antiferroelectric ordering, are generally desired.

Any discussion, including discussion of problems and solutions, set forth in this section, has been included in this disclosure solely for the purpose of providing a context for the present disclosure, and should not be taken as an admission that any or all of the discussion was known at the time the invention was made or otherwise constitutes prior art.

### SUMMARY OF DISCLOSURE

Various embodiments of the present disclosure relate to devices including nematic liquid crystal-forming fluid and to method of forming an using such devices. In accordance with examples of the disclosure, the nematic liquid crystal-forming fluid includes molecules including one or more dipoles, wherein the one or more dipoles exist in a ferroelectric nematic state. This allows devices to operate with ferroelectric characteristics.

In accordance with various examples of the disclosure, a variety of devices including ferroelectric nematic liquid crystals are provided. Exemplary devices include molecules with desired molecular orientation and polarity that are obtained by using ferroelectricity to achieve a relatively high coupling to an electric field within a volume comprising ferroelectric nematic liquid crystal-forming fluid and to relatively high charge within their volume, and which consequently exhibit unprecedented electro-optical and electro-mechanical responses. These strong responses can be highly geometry specific. For example, the polar ordering direction of the fully aligned dipoles near, e.g., glass surfaces of cell plates, can strongly prefer to be parallel to the cell plates, so their facile reorientations are about the normal to the plates. These reorientations can be induced by electric fields applied to be parallel to the cell plates. Thus embodiments of the present disclosure relate to the geometrical arrangement and manipulation of the ferroelectric polarization of the volume.

In accordance with examples of the disclosure, a device includes a volume comprising ferroelectric nematic liquid crystal-forming fluid and means for containing said fluid. The fluid includes molecules having one or more electric dipoles, said molecules having spontaneously formed a ferroelectric polarization density, said spontaneous polarization density comprising a nonzero local unidirectional average orientation of said dipoles, and said polarization density comprising a magnitude and a vectorial direction in said volume. In accordance with various aspects of these embodiments, the device can be used for electrical control of an electromagnetic field. In this case, the device can include one or more electrodes for application of an electric field to said volume, and the electromagnetic field propagates in said volume, said electric field causing said polarization density to change in magnitude, thereby producing a change in the electromagnetic field. In accordance with additional aspects, the device can be used for electrical control of an electromagnetic field. In this case, the device can include one or more electrodes for application of an electric field to said volume, and an electromagnetic field to be controlled propagates in said volume, said electric field causing said polarization density to change the vectorial direction, thereby producing a change in the electromagnetic field. In accordance with yet additional aspects, the device can be used for producing electrically-driven motion. In such cases, the device can include one or more electrodes for application of an electric field to said volume, said electric field causing said polarization density to change in the vectorial direction and/or the magnitude, thereby producing a physical motion of or change of shape of said volume. In accordance with other aspects, the device can be used for performing mechanical sensing, wherein said device includes one or more electrodes for measuring the electric potential or current flow within said volume, said electric potential and/or current flow generated by change in said polarization density, said change due to a variation in stress within said volume or change of shape of at least a portion of said volume. In accordance with yet additional aspects, the device can be used for thermally generating a charge density, wherein said device includes one or more electrodes for measuring an electric potential or obtaining a current flow within said volume, said electric potential and/or current flow generated by a change in said polarization density, said change of said polarization density produced by a change in temperature of said volume. In accordance with further examples, the device can be used for performing molecular dipole scavenging, wherein said polarization density produces local molecular scale cavities, said cavities binding molecules having dipoles in said volume.

The means for containing said fluid can include, for example, (e.g., parallel and/or planar) surfaces, such as plates or the like. The electric field can be applied parallel to one or more of the surfaces. The polarization density can be parallel to one or more of the surfaces. The electromagnetic field can have a polarization parallel to one or more of the surfaces. The electric field, polarization density, and a polarization of said electromagnetic field can be along the same line. The electromagnetic field can include one or more of microwave, infrared, visible, ultraviolet, and x-ray light, propagating in or reflecting from said device.

In accordance with further examples, the molecules comprise features suitable for the stabilization of a ferroelectric nematic phase comprising one or more of (1) a rod shape having a molecular long axis suitable for nematic liquid crystal ordering; (2) a substantial molecular net dipole parallel to the molecular long axis, said dipole stabilizing head-to-tail chaining of said rod-shaped molecules; (3) molecular subcomponents along the molecular length giving localized charges of alternating sign distributed along said molecular long axis; (4) minimal flexible tails to enable dipolar charges to interact, but provide enough flexibility to suppress crystallization; and (5) lateral groups to control the relative positions along the director of side-by-side molecules, to promote their polar order.

In accordance with further examples of the disclosure, a device for electrical control of an electromagnetic field is provided. The device includes a volume comprising nematic liquid crystal-forming fluid. As above, the fluid includes molecules comprising one or more dipoles, said one or more dipoles existing in a ferroelectric nematic state. The ferroelectric state can include, at places within said volume, a macroscopic electric polarization density with an average local unidirectional polar ordering. The ferroelectric nematic liquid crystal will acquire an electric potential energy due to said dipole ordering in response to application of an electric field, the gradient of said potential applying force and torque to said dipoles causing the dipoles to change orientation. The orientation change can produce a change in the electromagnetic field.

In accordance with further examples of the disclosure, a device includes nematic liquid crystal-forming molecules including one or more dipoles, wherein the dipoles exist in a ferroelectric nematic state; and one or more electrical connections to apply an electric field to the nematic liquid crystal-forming molecules.

In accordance with further examples, the device includes one or more (e.g., parallel) plates, wherein at least one of the plates comprises electrodes to provide or form the electric field.

In accordance with further examples of the disclosure, an electromotive device is provided. The device includes a volume comprising nematic liquid crystal-forming fluid. The fluid includes molecules possessing one or more electric dipoles, said one or more dipoles existing in a ferroelectric nematic state, said state having, at places within said volume, a macroscopic electric polarization density with an average local unidirectional polar ordering. The ferroelectric nematic liquid crystal acquires an electric potential energy due to said dipole ordering in response to application of an electric field, a gradient of said potential applying force and torque to said dipoles causing the dipoles to produce motion or change of shape of said liquid crystal volume.

In accordance with further examples of the disclosure, means for containing said fluid include one or more surfaces, such as one or more surfaces described herein.

In accordance with further examples, the molecules include a positive charge at one end and a negative charge at the other end. In accordance with further examples, the molecules can include about 2 to about 5 cyclic structures, such as C6 cyclic structures. Additionally or alternatively, the molecules can include one or more acetate functional groups. In some cases, the molecules can include methoxy and/or nitro functional groups—e.g., respective ends of the molecules. In some cases, the molecule includes two methoxy groups.

In accordance with various examples of the disclosure, the electric field is less than 1 V/cm or between about 1 mV/cm and about 1 V/cm.

In accordance with additional examples of the disclosure, the ferroelectric nematic fluid comprises dimeric, oligomeric, or polymeric material.

In accordance with additional examples of the disclosure, the ferroelectric nematic fluid comprises or is a glass, or may exhibit a glass transition.

In accordance with additional examples of the disclosure, the ferroelectric nematic fluid comprises or is elastomeric material.

In accordance with additional examples of the disclosure, the ferroelectric nematic fluid comprises or is viscoelastic material.

In accordance with additional examples of the disclosure, the ferroelectric nematic fluid exhibits a yield stress.

In accordance with further embodiments of the disclosure, a method of using a device as described herein is provided.

In accordance with further examples, a method is provided for discovering molecular structures with features suitable for stabilization of the ferroelectric nematic phase, said method comprising atomistic molecular dynamic computer simulation, said simulation achieving thermal equilibration of at least two samples of a number of test molecules, said test molecules having a molecular dipolar structure, one of said samples comprising a polar collection of test molecules initiated with maximum polar order of said dipoles, and another one of said samples comprising a nonpolar collection of test molecules initiated with zero polar order of said dipoles, said method comprising the determination and comparison of the mode of forming of polar intermolecular correlations in the polar and nonpolar systems.

### BRIEF DESCRIPTION OF THE DRAWING FIGURES

FIG. 1 illustrates ferroelectric nematic phase of a volume in accordance with examples of the disclosure. Panel (A) illustrates a structure of a compound 1 (suitable for the molecules) and schematic of molecular alignment in the ferroelectric nematic (NF) phase. The molecular organization is translationally symmetric in 3D and macroscopically uniaxial, with local mean molecular long axis, n(r), aligned generally along the buffing direction z, and polar, with a local mean molecular dipole orientation, P(r) along n. H and O will be used to represent the methoxy and nitro-ends of the molecule respectively. Panels B-G illustrate DTLM images showing electro-optic evidence for ferroelectricity in a planar-aligned cell of compound 1 in the NF phase (t=11 μm thick), with an in-plane field, E, applied along the buffing direction as indicated. In the higher temperature N phase, P(r)=0, but when cooled into the NF phase without an applied field, compound 1 spontaneously forms macroscopic domains with P>0 or P<0. Panels B-D illustrate coarsening process upon cooling in the NF phase ending in a pattern of domains with distinct boundaries. Panels E and G illustrate application of an ultra-small field, E˜1V/cm, results in-plane reorientation of n(r), producing the dark bands either inside or outside of the domains, depending on the sign of E. The E˜1V/cm threshold field for this reorientation indicates that n(r) in these domains is coupled to E by a polarization P˜6 μC/cm2, which is comparable to the bulk polarization density measured electronically. (F) Higher applied field moves the domain boundaries to increase the area of the field preferred orientation, affecting a hysteretic reversal of P(r). Scale bar=100 μm. Arrows indicate the flow of the caption.

FIG. 2 illustrates DTLM images showing polar Freedericksz twist transition threshold in ferroelectric domains with opposite polar orientation. (A) Field-free initial state showing three domains separated by domain walls, each domain having n(r) along the rubbing direction. (B) Application of Ez>0 induces a birefringence color change resulting from in-plane reorientation of n(r) in the upper and lower domains. There is little optical change or reorientation in the central domain. If the field is returned to E=0, the system returns to the starting state (A). (C) Application of Ez<0 induces an in-plane reorientation of n(r) in the center domain, leaving the upper and lower domains unchanged. These observations demonstrate the polar asymmetry of the domains and also enable the absolute determination of the direction of P(r): the domain having the preferred orientation in the applied field doesn't reorient. In this experiment, the field is not large enough to move the domain walls, which are pinned by the surfaces. The polarization vectors 202 indicate the field-induced reorientation of P(r) in the mid-plane of the cell: the polarization does not reorient at the surfaces in this experiment, remaining parallel to z. These field-induced reorientations with P(r) starting nearly antiparallel to E are polar azimuthal Freedericksz transitions. The threshold field, EP=(π/t)2(KT/P), estimated using the measured P˜6 μC/cm2 from FIG. 3, is EP˜1 V/cm, comparable to the fields employed here. This agreement indicates that, in absence of applied field, these domains have formed with the full P˜6 μC/cm2 polarization. t=11 μm. Scale bar=100 μm.

FIG. 3 illustrates Characteristics of polarization reversal by field. (A) Dependence on T of the cell current with a 200 Hz square wave of peak field Eₚ=95V/mm amplitude applied in-plane to a t=15 μm thick cell with 1 cm wide ITO electrodes spaced by d=1 mm. In the I and N phases (T>=133° C.), the current is small and capacitive, while upon cooling into the N_{F} phase an additional current peak appears, the area of which is independent of voltage and is equal to the net polarization charge reversal, Q=2PA, where A=15 μm×1 cm is the effective cross-sectional area of the volume of LC material reoriented by the in-plane applied field. In the N_{F} phase, this peak grows in area on cooling, indicative of an increasing polarization density, and also becomes longer in time, reflecting the increase of orientational viscosity. The dark arrow shows the reversal time at T=120° C. [dotted lines in (C)]. (B) black squares—The polarization density P of compound 1 obtained on cooling saturates at P˜6 μC/cm² at the lowest temperatures. The region near the transition has not been studied in detail. C) Field dependence of the time Δt, the half-width at half-height of the polarization or optical reversal pulse induced by field steps of a 100 Hz square wave of peak amplitude Ep in planar-aligned cells with in-plane electrodes spaced by d=20 μm, 60 μm, and 1 mm at T=110° C. The reversal times scale as 1/Eₚ as expected for reorientation driven by ferroelectric P×E torques. The dotted lines show the data for Eₚ=95 V/mm [dark arrow in (A)]. The risetime τ=1/PE is ˜0.1Δt.

FIG. 4 illustrates DTLM images of a domain (402, 404, or 406), having surface polarization to the right, shrinking in a dark background of field-preferred surface and bulk polarization to the left. (A) Domain and structural elements P, n, and E). Dark spots appear where the surface disclination lines overlap (circles), making the π director reorientation uniform along x through the cell. Scale bar=50 μm. (B) Cross-section showing the 2D structure of P(r): the uniform (U) field preferred state; the initial domain being reversed, with the rotation of P in the center forming a twisted-untwisted (TU) state indicated by vectors 408; the intermediate twisted states, right handed olive (T_{R}) and left handed gold (T_{L}). The section drawing gives the 2D structure of the domain in the x,z plane along the top edge of the image in (B), showing π surface disclination lines (dots 410) mediating the polarization reorientation at the top (line 412) and bottom (line 414) cell plates. (C) Shrinking of the initial domain, with E increasing from left to right, successively increasing the rotation of P in the center domain and thereby changing its birefringence color. (A) Dark spots appear where the surface disclination lines overlap (circles), making the π director reorientation uniform along x through the cell. t=11 μm. Scale bar=50 μm.

FIG. 5 illustrates common polarization reversal scenarios in compound 1. Vectors 502 indicate field-induced reorientations. (A) Stripe formation. Applying a 5 Hz triangle wave reversal field of peak amplitude in the range 0<Ep<10V/cm to a region with an initially uniform in-plane director induces a periodic modulation in the orientation of n(r) and P(r) along z, a director bend wave. As the applied field strength is increased, the stripes have uniform internal orientation determined by the field strength (white arcs) and sharp boundaries. The herringbone arrangement of the director in the stripes ensures that the normal component of P is constant across the stripe boundaries, so that there is no net polarization charge on them. (B) Polygonal domains. During field reversal, polarization charge effects lead to the formation of a tiling of domains of uniform n(r). These polygons have sharp domain boundaries that are oriented such that P·l, where l is along the boundary (panel 4) is the same on both sides of the boundary, reducing space charge. (C) Director field around inclusions. Air bubbles in the cell can be used to track the orientation of n(r) in a reversing field. The director field near the bubble, sketched below each panel, is locally distorted, bending around the inclusion with splay deformations confined to two 180° wedge disclinations (which can show as red dots) at the ends of the bubble. A blue color was visible in panels 4 and 5 and corresponds to a TU state of the kind shown in FIG. 4, with a surface disclination then moving out from the bubble boundary to give the uniform state seen in panel 6. Scale bars: A=100 μm; B=100 μm; C=50 μm.

FIG. 6 illustrates ferroelectric nematic field-induced flow. (A) DTLM image of a t=10 μm thick, planar-aligned cell of compound 1 between untreated glass plates, in the NF phase at T=120° C. The black bars at the bottom are two evaporated gold electrodes 602 on one of the plates, separated by a d=60 μm gap. The electrodes 602 are outlined in white for clarity. Only the upper edges of the electrodes and cell are shown. A Vp=3V, 0.1 Hz square-wave voltage is applied to the electrodes, producing an electric field in the plane of the cell. This field drives a pattern of defect motion and fluid flow over the entire image, with the defect velocity v(r) (arrows 604) vectorially parallel to the applied field, E(r), which is tangent to half circles centered on the electrode gap. Where the defects are dense, their motion transports the surrounding fluid. When the field is on, the entire region shown here moves along the field lines. This image was captured at the instant of field reversal, where the resulting polarization reversal generates a periodic array of bend domain walls normal to the director and the field, as in FIG. 5, panel A, in this case along radial lines. (B) Typical defect in the texture moving along the applied field direction (down on the left, up on the right), in the location circled in (A). (C) Temperature dependence of the magnitude of the initial defect velocity along the white dashed track following a field reversal. A similar dependence on T is obtained on heating or cooling through the N-NF transition. In the NF phase the velocity increases with increasing P near the transition but decreases at lower T because of the increasing viscosity. Scale bars: A=1 mm, B=100 μm.

FIG. 7 illustrates exemplary geometries for exploiting the polarization of a ferroelectric nematic. (A) Using a spatially nonuniform polarization; (B) Inducing a nonuniform polarization; (C) Reorienting the ferroelectric polarization.

FIG. 8 illustrates instantaneous configuration from the POL MD simulation system of RM734 at T=130° C., employing a polarizable molecular model. The system was initialized in a polar state, and equilibrated retains a high degree of nematic and polar orientational order, as end-to-end flips are not observed. The vertical cell dimension is 70 Å.

FIG. 9 illustrates geometry-optimized structure of RM734 computed at the B3LYP/6-31G* level of theory, showing the orientation of the 11.4 D molecular dipole moment (arrow) for this specific molecular conformation. Other low-energy conformations have comparable dipole moments.

FIG. 10 illustrates static site charge distribution used in the atomistic simulations. The overall charges of specific functional groups, indicated in large type, show an alternation of group charges along the length of the molecule. The dashed lines correspond to lone-pair electrons.

FIG. 11 illustrates decomposition of the static site charge distribution into group dipole contributions. Irreducible bond and ring dipole moments are shown as small arrows, where the numerical value is the dipole moment in Debye (D). The dipole moments of specific functional groups are also indicated (large arrows and large, non-italic text). The numbers in italics are the average contributions of specific functional groups to the computed ferroelectric polarization density P_{S}, in units of μC/cm². The nitro group and the ring to which it is attached (the nitro ring) have the largest dipole moments, and together contribute 64% of the total polarization density. Four functional groups (nitro, nitro ring, central ring, and terminal methoxy) contribute 90% of the total polarization density. The ester groups and lateral methoxy possess substantial lateral dipole moments, which may contribute to intermolecular association.

FIG. 12 illustrates results of atomistic molecular dynamic simulations designed specifically to explore the molecular interactions and resulting positional/orientational correlations responsible for the polar molecular ordering of RM734, shown in (A). A nanoscale volume containing 384 molecules is equilibrated in these simulations into two distinct LC states: a POLAR system with all polar molecular long axes, u, along +z, and a NONPOLAR system with half along +z and half along −z. Equilibration of the molecular conformation and packing is readily achieved but end-to-end flips are rare so the equilibrated states remain in the limit of polar or nonpolar nematic order, respectively. (B,F,G) The POL simulation shows directly the dominant pair correlations adopted by molecules that are polar ordered, in the form of conditional probability densities, g(ρ,z), of molecular centers around a molecule with its center at the origin and long axis u along z. The g(ρ,z) are φ-averaged to be uniaxially symmetric, reflecting the uniaxial symmetry of the N and N_{F} phases. They exhibit a molecule-shaped, low-density region (g(ρ,z)˜0) around the origin resulting from the steric overlap exclusion of the molecules; an asymptotic constant value at large p giving the normalized average density (g(ρ,z)=1); and distinct peaks indicating preferred modes of molecular packing. This analysis reveals two principal preferred packing modes in the POL system: (B,F) polar head-to-tail association stabilized by the attraction of the terminal nitro and methoxy groups, and (B,G) polar side-by-side association governed by group charges along the molecule, nitro-lateral methoxy attraction, and steric interactions of the lateral methoxys. (D,E) The NOPOL system exhibits distinct correlation functions for antiparallel and parallel molecular pairs, g_{NP}^{anti}(ρ,z) and g_{NP}^{par}(ρ,z). (E,H,I) The preferred antiparallel packing gives strong side-by-side correlations, governed by group charges along the molecule; and (E,J,K) weaker antipolar nitro-nitro end-to-end association. (D,F,G) The parallel correlations in the NONPOL system are the most relevant to the stability of polar order in the N_{F} phase, as they are determined by the inherent tendency of the molecular interactions for polar ordering in the presence of enforced polar disorder. Comparison of (B) and (D) shows identical preferred modes of parallel association in the two systems, with the POL system correlations being even stronger in the NONPOL system. This is clear evidence that the polar packing motifs giving the correlation functions (B) and (D), exemplified by the sample POL MD configurations (F) and (G), stabilize the polar order of the ferroelectric nematic phase.

FIG. 13 illustrates a device in accordance with various examples of the disclosure.

### DETAILED DESCRIPTION OF EXEMPLARY EMBODIMENTS OF THE DISCLOSURE

The description of exemplary embodiments provided below is merely exemplary and is intended for purposes of illustration only; the following description is not intended to limit the scope of the disclosure or the claims. Moreover, recitation of multiple embodiments having stated features is not intended to exclude other embodiments having additional features or other embodiments incorporating different combinations of the stated features.

Examples of the disclosure provide improved liquid crystals and ferroelectrics, creating new opportunities for applications in both fields, especially in the areas of: (i) electric field control of optical properties; (ii) generation of electric field by applied strain and/or stress (piezoelectricity); (iii) generation of electric field by temperature change (pyroelectricity); (iv) electric field generation of stress, strain, and flow (electrohydrodynamics); (v) polar response to applied optical electric fields (nonlinear optics and electronic electro-optics), and the like. Further examples of the disclosure relate to devices including such materials, to methods of using such device, and to methods of forming the devices.

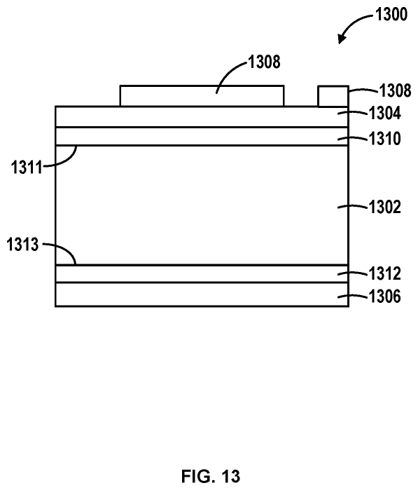

FIG. 13 illustrates a device 1300 in accordance with various examples of the disclosure. Device 1300 includes a volume 1302 comprising ferroelectric nematic liquid crystal-forming fluid and means (e.g., plates or surfaces 1304, 1306) for containing said fluid. The plates or surfaces can include, for example, glass, polymers, such as PET, polycarbonate, or the like. In the illustrated example, device 1300 also includes one or more polymer layers 1310, 1312. Exemplary polymers for layers 1310, 1312 include polyimide. Surfaces 1311 and/or 1313 can be buffed, using velvet, for example.

As set forth in more detail below the fluid can include molecules having one or more electric dipoles, said molecules having spontaneously formed a ferroelectric polarization density, said polarization density comprising a nonzero local unidirectional average orientation of said dipoles, and said polarization density comprising a magnitude and a vectorial direction in said volume. Device 1300 can be used for a variety of applications, such as the applications noted herein.

Exemplary molecules for various devices and application set forth herein can include, for example, (1) a rod shape suitable for nematic liquid crystal ordering; (2) a substantial molecular net dipole parallel to the molecular long axis, said dipole stabilizing head-to-tail chaining of said rod-shaped molecules; (3) molecular subcomponents along the molecular length giving localized charges distributed along the molecular long axis, said charges interacting with opposite charges; (4) minimal flexible tails to enable dipolar charges to interact, but provide enough flexibility to suppress crystallization; (5) lateral groups to control the relative positions along the director of side-by-side molecules, to promote their polar order. The extremely broad potential palate of synthesizable organic molecules possessing these properties enable the use of a variety of ferroelectric nematic molecules.

By way of example, the molecules can include 4-[(4-nitrophenoxy)carbonyl]phenyl2,4-dimethoxybenzoate (compound 1), a rod-shaped molecule with a large electrical dipole moment parallel to its long axis. Using preparations described herein, it was found that this compound exhibits 4 distinct phases: isotropic fluid (I) −188° C.—nematic fluid (N) −133° C.—ferroelectric nematic fluid (FF) −70° C.—crystal (X). Here the temperatures indicate where the transitions between the different phases occur. Thus we have found that this molecule makes a ferroelectric fluid (FF) upon cooling in the temperature range 133° C.>T>70° C.

Electro-Optics—Observations were made using Depolarized transmission light microscopy (DTLM) on cells with the material in a gap of width t between glass plates, one of which was coated with a pair of planar ITO electrodes 1308, e.g., uniformly spaced, which enabled application of an in-plane electric field, E, between them, largely parallel to the (x,z) cell plane. The plates were treated with a polymer layer 1310, 1312. Surfaces 1311 and 1313 were buffed in the z direction, normal to the electrode gap, so that the applied field was along the buffing direction: E=zE. The cells were filled by capillarity with the material in the isotropic phase T>188° C. Both the N and FF phases were studied, with results as follows.

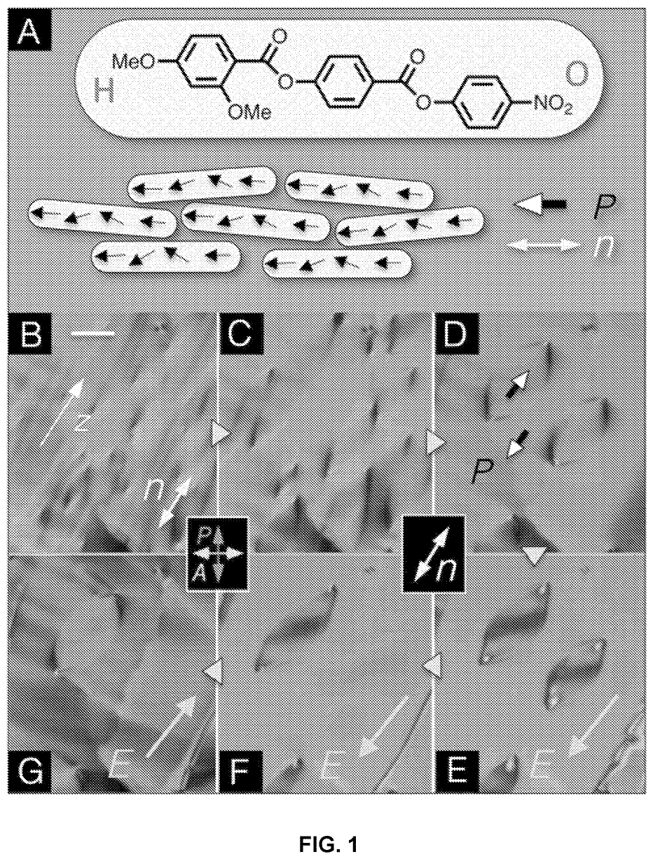

The key evidence for this result is our first observation in any fluid of the defining characteristics of ferroelectricity: (i) the formation, in the absence of applied electric field, of spontaneously electrically polar domains of opposite sign of polarization separated by distinct domain boundaries; and (ii) electric field-induced polarization reversal mediated by movement of these domain boundaries. This observation, summarized in FIG. 1, has enabled us to identify this fluid as ferroelectric and thereby discover a variety of novel physical behaviors of ferroelectric fluids.

In the N phase, the local texture of the planar-aligned cell illustrated in FIG. 1 is optically featureless. On cooling toward the FF phase, a random pattern of stripes extended along the buffing direction appears, but once in the FF phase these stripes coarsen, leading to a texture that is again local optically featureless (panels B-D) but characterized on a larger scale by a pattern of well-defined lines, some delineating distinct, lens-shaped and extended linear domains 100 μm or more in extent (panels D-G), all formed in the absence of applied electric field. Application of an ultra-small (˜1 V/cm), in-plane, DC test field, E, applied parallel to the in-plane buffing and director n, shows that for E>0 the director inside these domains begins to reorient while the orientation outside remains fixed (panel E), whereas for E<0 the region outside the lens-shaped domains reorients and the orientation inside remains fixed (panel G), indicating that the domain boundaries separate regions with opposite response to in-plane field, and therefore of opposite in-plane polarization. Increasing the field causes the domain boundaries to unpin, shrink, and disappear (panel F), moving hysteretically to increase the area of the field-preferred state. As E is increased, the polarization P(r) in the preferred state must become increasingly well-aligned along the field. We find optically that n(r) on one side of a given domain boundary does become better aligned along E (see, for example, the outside of the lenses in panel E and the inside of the lens in panel G) but that this effect is small, evidence that these are regions where P is already nearly parallel to E, and, importantly, since n is along E, implying that n is either parallel or antiparallel to P in the FF phase. These observations, constituting a first-principles demonstration of nematic ferroelectricity, are described in more detail next.

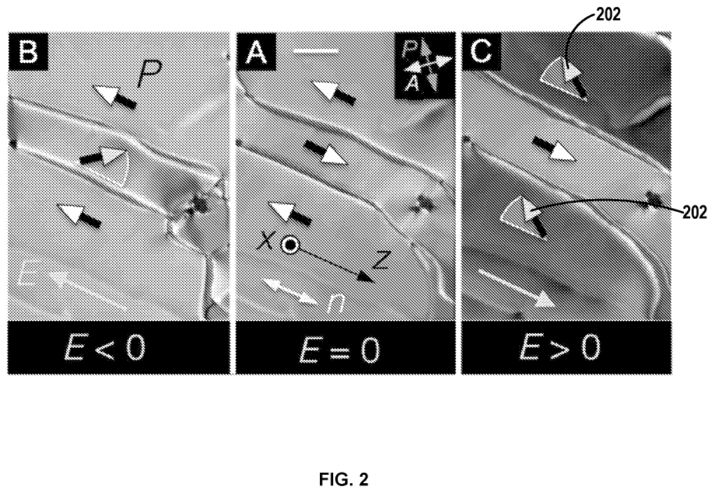

Nematic (N) Phase—When cooled into the Nematic phase, compound 1 formed textures with the nematic director, n(r), the local mean molecular long axis and the optic axis, parallel to the plates (planar alignment), as indicated by a birefringence Δn˜0.2. An azimuthal twist reorientation of n(r) across the thickness of the cell can be induced in the N phase in this planar-aligned geometry using an in-plane 1 kHz AC field with E˜1000 V/cm. This field strength is comparable to that estimated for the dielectric Freedericksz threshold field E_{D}=(π/t)√K/εₒΔε), assuming a cell gap t=11 μm, a Frank elastic constant K˜5 pN, and a dielectric anisotropy Δε˜5. This ED sets the field scale for typical in-plane dielectric nematic electro-optics.
Ferroelectric Nematic Fluid (FF) Phase—Upon cooling through the N-FF transition, the cell becomes patterned with a texture of irregular domains extended locally parallel to n(r), first appearing on a submicron scale but then annealing over a roughly 2° C. interval into a pattern of elongated lines of low optical contrast that are and also oriented generally along n(r). These lines coarsen to form closed loops, 10-200 microns in extent, having a distinctive characteristic lens shape, elongated along n(r). Sample textures from this evolution are shown in FIG. 1. Upon completion of the transition, apart from these loops, the texture is smooth and very similar to that of the N phase. The FF phase exhibits striking electro-optic behavior in DTLM, starting with the response to the application of tiny (<1 V/cm) in-plane electric fields, four orders of magnitude smaller than the threshold for dielectric response in the N phase. This sensitivity was exploited to probe and understand the static and dynamic changes of n(r) and P(r) in applied electric fields. Typical textures observed in these experiments and the responses used to identify ferroelectric domains and determine their polarization orientation are indicated in FIG. 2. In the absence of applied field, the LC director n in these cells is generally along the buffing direction z and we observe domains separated by distinct boundaries. As in FIG. 1, one set of domains responds only weakly to fields applied along z, indicating that in these regions P is already parallel to E, and therefore that before the field is applied P is everywhere either nearly parallel to or antiparallel to z. In an applied field, in the domains where P is nearly antiparallel to E, the polarization responds by rotating toward E. The optical response to test fields makes the difference in polarity readily distinguishable, leaving no doubt that these domains are polar. Vectors in FIG. 2 indicate the field-induced reorientation of P(r) in the mid-plane of the cell: n(r) at the cell surfaces in this experiment remains parallel to z. These field-induced reorientations are polar Freedericksz transitions which have a threshold field E_{P}=(π/t)²(K_{T}/P). Estimating the experimental threshold to be E_{P}˜1 V/cm, and K_{T}˜5 pN, we can estimate the corresponding spontaneous polarization to be P˜6 μC/cm².

With crossed polarizer and analyzer and the polarizer along the director n, these textures of n(r) in the limiting states of plus or minus E are identically or substantially similarly black, but separated by a striking scenario of domain wall formation, coarsening and disappearance, all in the tiny DC field range −2 V/cm<E<2 V/cm. The field-aligned states extinguish between crossed polarizers, meaning that that they have n(r) everywhere parallel to z, and show a (e.g., pink) birefringence color for white light incident. The intermediate states have n(r) in the y,z plane but with spatial variation of its azimuthal orientation φ(r) about x. This lowers the effective retardance of these regions, moving their birefringence down into the second- and first-order Michel-Levy bands and producing intense birefringence colors. The uniformly oriented domains obtained following field reversal are states in which the n,P couple has been reoriented in the bulk LC and also flipped on the aligning surfaces, the latter mediated by domain wall motion. Thus, in the temperature range of the FF, we can make device-size domains having a uniform orientation of dipole moment density P, controllable by in-plane electric fields of magnitude E less than 2 V/cm. This kind of sensitivity of molecular orientation in a nematic liquid crystal in a micron-gap cell to applied electric field is completely unprecedented in nematic liquid crystal science, including all previous studies of compound 1 and similarly structured liquid crystal forming rod shaped molecules.

In order to explore further this extreme sensitivity to field, we also measured directly the field-induced polarization density of the FF using the current associated with the field-induced reversal of the polarization. We used both square- and triangle-wave driving fields in several different two-terminal cell geometries with conventional gold or ITO electrodes; as well as a 0.5 mm diameter cylindrical capillary with the material in a 150 μm gap between planar electrode faces noal to the cylinder axis. The current signal obtained using these geometries exhibited a distinct current bump that disappeared for T>133° C., and produced consistent values of its time integral to give the polarization density as a function of temperature. The resulting P increases with decreasing T continuously from small values at the transition, saturating at low T at a value P˜6 μC/cm². The significance of P˜6 μC/cm² can be appreciated by calculating a polarization estimate Pₑ=p/v, where p=11 Debye is the axial molecular dipole moment of compound 1 and v is the volume/molecule in the phase, v=325 cm³/mole=540 Å³/molecule, assuming a LC mass density of ρ=1.3 g/cm³. Using these parameter values and assuming complete polar ordering of the molecular long axes, we find Pₑ˜6.7 μC/cm², comparable to our measured P at low T and indicating that the compound 1 FF has extremely strong spontaneous macroscopic polar ordering. This magnitude of polar ordering confirms our statement that we have achieved in our preparations a state in which the molecular axes are optimally aligned in a mutually parallel and polar fashion and that the FF state is a liquid crystal. Specifically, this result, combined with our optical microscopic observations, indicate that the FF is a 3D, macroscopically homogeneously polar, uniaxial nematic. The agreement with the P measured from the polar twist threshold indicates that this is the ferroelectric state achieved in our samples, as grown without a field. Our cell geometry enables one to uniquely take advantage of this polar ordering, as evidenced by the unprecedentedly strong coupling of molecular reorientation to applied field.

Given these very large polarization values, it is useful to summarize several of the relevant features of polar electro-optic, electrostatic, and elastic behavior, developed in these findings of chiral smectic ferroelectric LCs, which can now be expected for the FF: (i) Polar Freericksz transition—We take the uniform equilibrium state to have P along z. In small applied fields, electrical torque on the director field τ_{E}=P×E comes from the coupling of field to polarization. Applying this coupling in the description of the twist Freedericksz transition, the equation describing the azimuthal orientation field φ(x) across the thickness of an otherwise uniform cell becomes K_{T}φxx+PE sin φ=0, and the field threshold for reorientation with P(r) starting antiparallel to E is given by EP=(π/t)²(K_{T}/P)˜1 V/cm, comparable to the fields being employed in FIGS. 1 and 2 are 10⁴ times smaller the dielectric threshold E_{D} estimated above. It is important to point out again that the experimental observation that field-induced rotation does start near E=E_{P} confirms that the spontaneous polarization appearing upon cooling in the ferroelectric domains is comparable in magnitude to that obtained by polarization measurements under conditions of field-induced polarization reversal. (ii) Boundary penetration—Solving Kφ(z)_{zz}+PE sin φ(z)=0 instead in the y,z plane, with the boundary condition φ(z=0)=0, using the one-elastic constant (K) approximation, and applying an electric field to stabilize φ(z)=180° at large z, a π reorientation wall is established in the LC near z=0, given by φ(z)=4 tan⁻¹[1−exp(−z/ξ_{E})]. The ferroelectric field penetration length ξ_{E}=√K/PE gives the approximate width of the wall, the distance that the effects of local orientational pinning such as the wall can penetrate into the neighboring LC, assuming the latter has P held in place by E. The penetration depth is ξ_{F}˜1 μm for an applied field E=1 V/cm). (iii) Block polarization reorientation and expulsion of splay (splay-elastic stiffening)—Spatial variation of P(r) generally results in bulk and surface polarization charge density, given respectively by ρ_{P}=∇·P(r) and σ_{P}=P_{S}·s. The electric field generated by the bulk charge opposes the bulk distortion of P(r) that caused it, producing a bulk energy

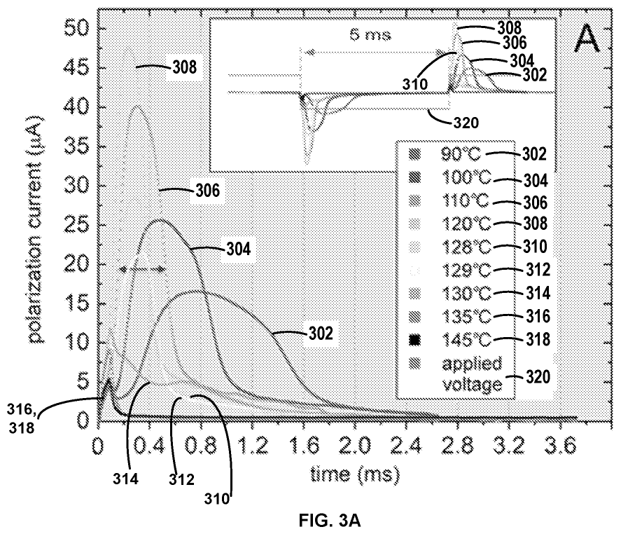

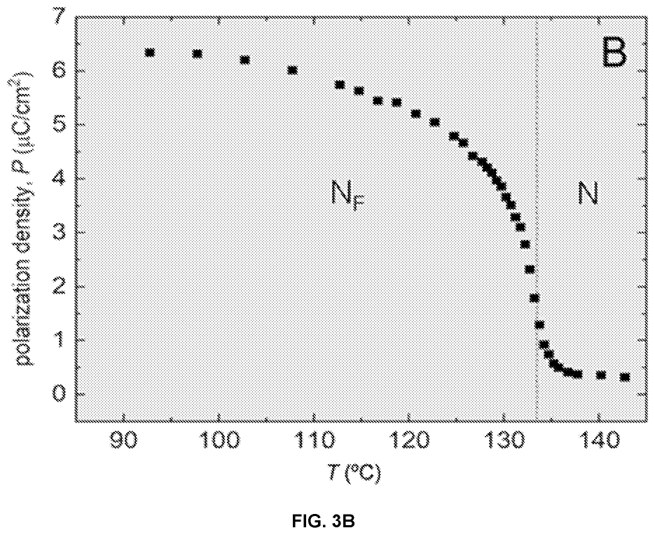

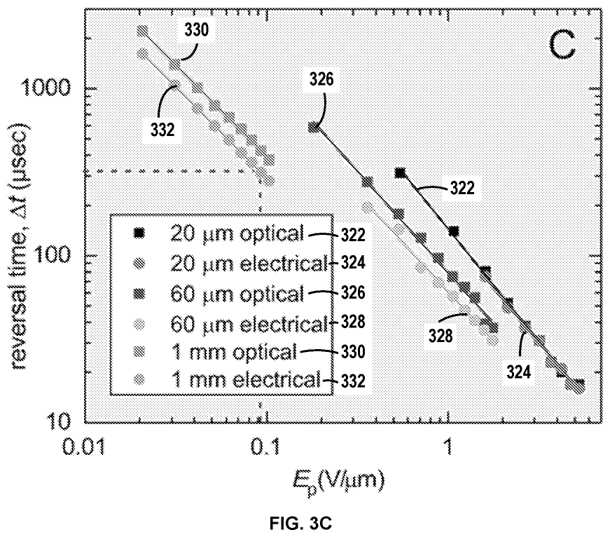

U_{P}=½fdv ∇·P(r)∇·P(r′)[1/|(r−r′)|]. Assuming a periodic transverse modulation δP_{y}(r) of amplitude Pδn_{y} and wavevector q_{y}, so that ∇·P(r)=∂P_{y}(y)y=iq_{y}P_{z}δn_{y} in our geometry, we have an elastic energy density Uₛₚ=½[Kₛq_{y}²+4πP²/ε]|δn_{y}|², meaning that the polarization term will be dominant for
q_{y}<π√2/ξ_{P}, where ξ_{P}=√εK/P² is the polarization self-penetration length. Since for P=6 μC/cm² we have ξ_{P}˜0.1 nm, this dominance will act down to molecular length scales. The result is that low-energy elastic distortions of the n,P couple allow only bend, with splay of n(r) and P(r) expelled from the bulk and confined to reorientation walls of characteristic width ξ_{P}. On the other hand, if we consider a longitudinal modulation δP_{z}(y,z), the additional electrostatic free energy density will be
U_{P}=½[4πP²q_{z}²/ε(q_{z}²+q_{y}²)]|δP_{z}|². All such polarization-based effects are reduced by free space charge, such as ionic impurities in the LC and its containing surfaces, ionization of the LC itself, and charge injected from the electrodes, all of which tend to screen the polarization charge. In SmC* FLC cells, when the polarization is small (P<20 nC/cm²) the bound polarization charge can be substantially screened but for large polarizations (P>100 nC/cm²) the free charge supply can be exhausted and polarization effects manifested. For the largest SmC* polarizations (P˜800 nC/cm²), the polarization charge is largely unscreened and the polarization effects are quite dramatic. (iv) Field-step reorientational response—The reorientation dynamics leading to polarization change and the electro-optic (EO) response to changes in applied field are complex, depending on elastic, viscous, surface, and flow-induced torques in addition to that of the field. However, with the application of a large field step the electrical torques initially dominate and these determine the risetime of the optical response. The balance of field and viscous torques gives a characteristic reorientation risetime on the order of τ=γ₁/PE, where γ₁ is the nematic rotational viscosity (FIG. 3, panel C).

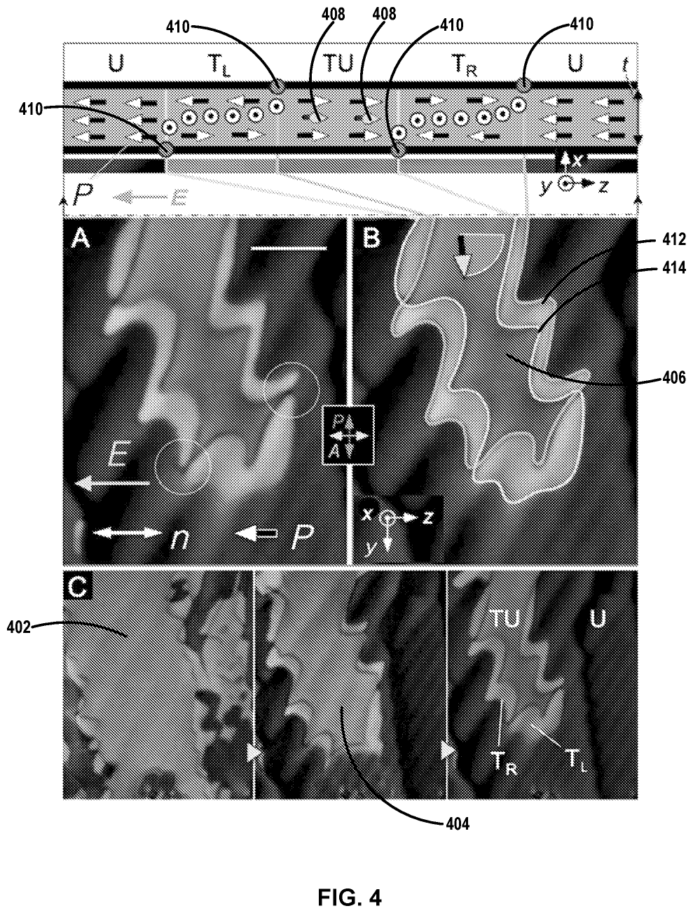

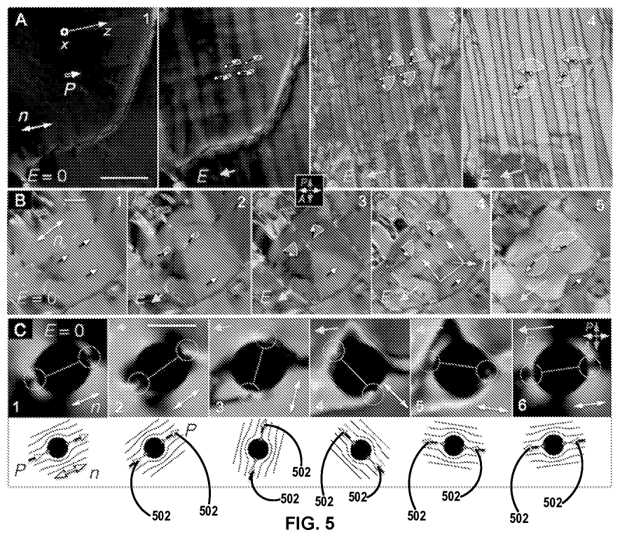

The electro-optic response of the FF phase in a cell with in-plane electric field applied shows uniquely polar features, with P(r) reorienting in the y,z plane through an azimuthal angle φ(r) determined by the local surface, elastic and electric torques. Buffed surfaces stabilize two planar-aligned states (φ=0 and φ=π) with opposite signs of P(r), so the cell has four stable states, two that are uniform and two that are twisted, illustrated in FIG. 5. These equilibrium states are separated by π surface disclination walls (magenta dots in the section drawing of FIG. 4). If complete polarization reversal is to be achieved by an applied field, P must be switched on both surfaces. We refer to domain boundaries such as these, where both n and P reorient but maintain a fixed relative sign, as Polarization-director Disclinations (PnDs). In a texture with only PnDs the local orientation of P(r) relative to n(r) will be either parallel or antiparallel everywhere. In FIG. 4, an applied field preferring the uniform (U) dark state is deforming a central domain that had polarization that was initially opposed to the field and is now partially rotated toward it (vectors 408). This central domain result is a twisted-untwisted (TU) state in which the director twists along x from the surface-preferred alignment parallel to z at one cell plate through azimuthal angle φ(x) to the field-aligned orientation in the midplane of the cell, and then twists back to the surface-preferred alignment on the other glass plate. The field causes that domain to shrink, moving and eventually eliminating the disclination walls in order to achieve complete polarization reversal. The motion of the walls on the two cell surfaces is different because the pinning strengths are different and spatially inhomogeneous, with the result that they do not remain in register, leading to the formation of the olive (left hand, T_{L}) and gold (right hand, T_{R}) twisted states seen surrounding the central domain, and where the central TU domain color (402, 404, 406) depends on the degree of field-induced reorientation of n,P in the sample midplane.

FIG. 5 shows several other modes of field-induced polarization reversal. The initial response of a uniformly aligned region to an increasing in-plane DC field in the range 0<E<2 V/cm that opposes the local polarization is to form a zig-zag modulation in the orientation of n(r) and P(r), illustrated in panel A in which the non-zero spatial variation is ∂n(r)/∂z, along the director, making it a bend wave. Bend has a lower polarization space charge energy cost than a splay wave (nonzero ∂n(r)/∂x), which would generate stripes parallel to n rather than normal to it. As the field strength is increased, the degree of reorientation increases and distinct boundaries appear between the half-periods of the modulation, separating stripes of uniform internal orientation. Fields of a few V/cm drive complete (+π, −π, +π, −π) reorientation of n, for which these boundaries become 2π walls, sub-optical resolution (˜ξ_{P}) in width. This process is even more dramatic with dynamic driving as illustrated in FIG. 5, panel A, which presents snapshots of alternating field reversals generated by a 5 Hz AC triangle wave field of peak amplitude E_{P}=3 V/cm. A few cycles after application of E the stripes become very regularly and narrowly spaced. The zig-zag pattern of the stripes indicates an overall structure where P_{z} is constant, ensuring that there is no net polarization charge at the stripe boundaries, and where the backflow induced in each stripe matches that of its neighbor. The field is not strong enough to reverse the surfaces in this case. In FIG. 5, panel B, polygonal domains are formed upon reversal in which charge-stabilized areas of uniform P are bounded by sharp domain boundary lines, each oriented along a vector l such that P·l has the same value on either side of the line, as in panel 4, where the angular jump in n(r) is 90°. This geometry reduces the net polarization charge on the line. Similar structures are found in high-P chiral smectic ferroelectrics and in ferromagnets. The textures of the charge-stabilized domains can also be employed to directly visualize the reorientation of P(r) under applied field, as shown in FIG. 5, panel C, where a circular air bubble enables tracking the polar orientation of n(r) during field reversal. The n(r),P(r) structures are sketched below each panel. The director is anchored tangent to the bubble surface, resulting in a director field that is largely bent around the bubble, with splay concentrated in two 180° wedge disclinations (red dots). The line connecting these defects is parallel to n(r) in the area surrounding the bubble.

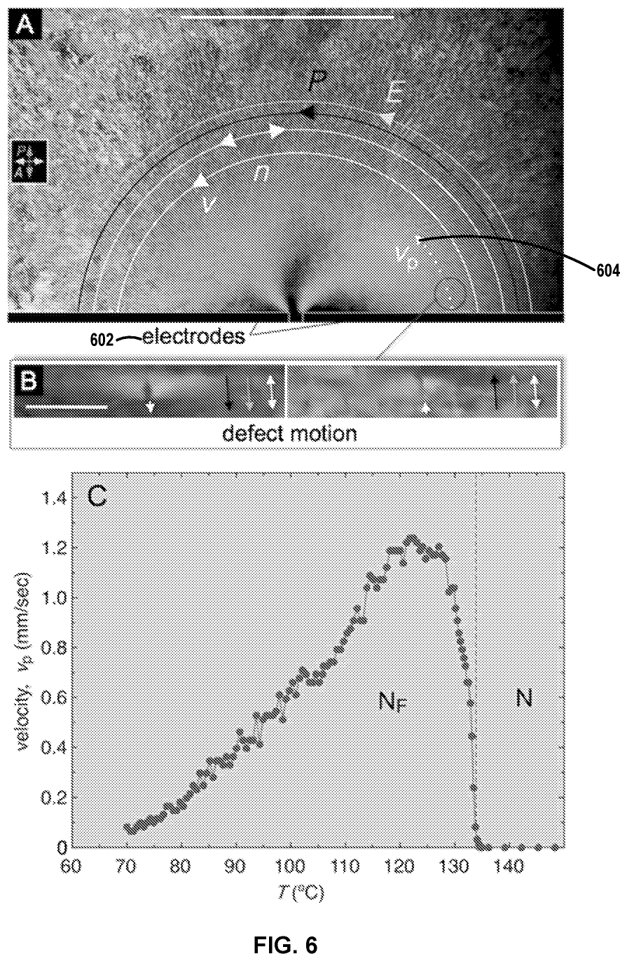

Ferroelectric Fluid electro-mechanics and -hydrodynamics—The polarization density of the FF phase creates a fluid which is extraordinarily responsive to both external applied fields and its internally generated polarization space charge. While the discussion above has focused on the effects of field-induced molecular reorientation, the most interesting and useful effects of the FF may be its ferroelectrohydrodynamic or ferroelectrorheological behavior, exemplified by the observations shown in FIG. 6. In this experiment, compound 1 is filled into a t=10 μm cell with random-planar alignment of n. An in-plane electric field is applied using a pair of gold electrodes evaporated with a d=60 μm gap onto one glass plate, visible at the bottom of FIG. 6(A). A square wave voltage with Vp=5 V applied between the electrodes generates an electric field distribution where E(r) is uniform in the gap and dipole-like in the surrounding area, directed along half-circles concentric with edge of the electrode gap. In the FF phase, this field induces flow of localized defects (FIG. 6(B)) and their surrounding fluid with a velocity field v(r,t) locally parallel to E(r), and reversing with the field, suggesting an electric body force density F(r)=ρ(r)E(r), where ρ(r) is a positive electric charge density. When E(r) goes through E=0 during field reversal, flow ceases and the director field breaks up into the P-reversal bend domain bands of FIG. 5A as it rotates alternately through +π and −π domains, giving the radial texture in FIG. 6A. Thus, dynamically P(r,t) is everywhere parallel to E(r,t) and v(r,t) when voltage is applied. The fact that the product P(r)·E(r) is unchanged by applied field reversal and yet v(r) changes sign, indicates that ρ(r) does not change sign with P(r), i.e., the driving has caused the fluid to become charged.

We measured vᵢ, the initial value of the defect velocity upon the field reversal at the location indicated in FIG. 6(A). This velocity depends dramatically on temperature, as shown in FIG. 6(C), with flow being essentially absent in the N phase and commencing upon cooling through the N-FF transition. The velocity eventually decreases with decreasing T, presumably because of the increasing viscosity of the LC. Thus, an applied electric field promotes the creation of regions with positive charge density.

Charging of the FF by AC field application is to be expected due to its polar asymmetry. Electrode surfaces contact FF material having the sign of P alternating in time. The FF, because of its polar symmetry, has diode-like polarity-dependent resistance that can also depend on the sign and nature of the charge carrier. The bulk charge mobility along z in the FF may also depend on field direction. Beyond this there will be a variety of charging effects due to the linear coupling of P and flow. If we consider, for example, steady, incompressible nematic laminar flow, then the director is generally nearly parallel to the velocity and v(r)=v(r)n(r). Since ∇·v(r)=0 we have ∇·n(s)=∂[ln v(s)]/∂s, where s is the position variable along flow: where the velocity increases the director splays inward. But in the FF phase we have P(r)=Pn(r), where P is the constant polarization, so that laminar flow produces polarization charge density ρ_{P}(s)=P∇·n(s)=P∂[ln v(s)]/∂s, the sign of which depends on whether P is aligned along v or opposed to it. Complex flows will thus produce complex patterns of polarization charge. Reorientation of P is displacement current, J=∂P/∂t, which is locally normal to P(r), and, if driven by electric field, gives a highly anisotropic contribution to the net electric conductivity, σ_{⊥}=P²/γ₁ for E⊥P, and σ_{∥}=0 for E∥P. For compound 1 σ_{⊥}˜10⁻³/Ωcm, which is in the semiconducting range. Under these circumstances, accumulation of one sign of charge in the fluid can occur when an applied AC field gets out of phase with polarization reversal. Additional inherent asymmetries, like differences in mobility or chemical character between positive and negative ionic impurities, or an intrinsic tendency for splay distortion of the P(r) field itself can also contribute.

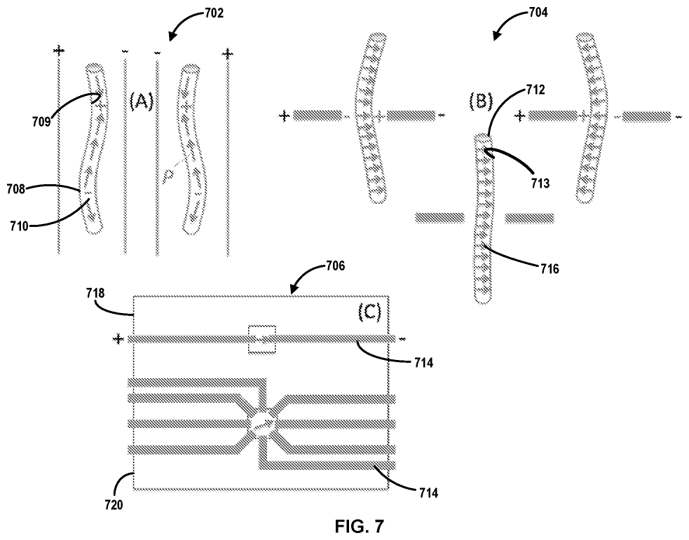

Nematic Piezoelectricity and Pyroelectricity—The ferroelectric nematic offers distinct advantages in applications requiring materials with a permanent macroscopic ferroelectric polarization, such as piezoelectricity and pyroelectricity. Cells with electrodes in contact with the FF show distinct charging of electrodes and generation of potential difference between electrodes in response to mechanical stress and/or temperature change. Pyroelectric charge generation is especially large upon cycling the sample temperature through the N to FF phase transition where ∂P/∂T is largest.
Example Geometries—FIG. 7 illustrate exemplary devices 702, 704, 706 including electrodes in accordance with examples of the disclosure. Devices 702-706 can be used in connection with any device and/or methods described herein.

FIG. 7 (A) illustrates a device suitable for mechanical driving. The device includes a (e.g., flexible) tube 708, having a surface 709, and a volume 710 comprising nematic liquid crystal-forming fluid within the tube. A field can be applied across the nematic liquid crystal-forming fluid, as illustrated, to cause or a field can be in response to a deflection in the tube. In these cases, an electromotive device can include a volume comprising ferroelectric nematic liquid crystal-forming fluid and means (e.g., one or more surfaces) for containing said fluid, said fluid comprising molecules having one or more electric dipoles, said molecules having spontaneously formed a ferroelectric polarization density, said polarization density comprising a nonzero local unidirectional average orientation of said dipoles, and said polarization density comprising a magnitude and a vectorial direction in said volume.

(B) illustrates a mechanical sensing. The mechanical sensing device can be used to, for example, measure a surface deflection. In these cases, the device can include a flexible tube 712, or sheet, having a surface 713 and nematic liquid crystal-forming fluid 716 within the tube or between the sheets. In these cases, a mechanical sensing device can include a volume as described herein, said ferroelectric nematic liquid crystal-forming fluid producing an electric potential and/or current flow in response to a stress or change of shape to at least a portion of the fluid.

(C) illustrates exemplary electrode configurations suitable for various embodiments of the disclosure. Device 706 includes at least one surface. The squares illustrate a volume 718 comprising nematic liquid crystal-forming fluid. The shaded areas correspond to electrodes 714, such as electrodes that can provide an in-plane electric field to the fluid.

Atomistic Molecular Dynamics (MD) Simulation—We carried out MD simulations directed toward gaining an understanding of how features of molecular architecture, interactions, and correlations are related to the polar ordering of the N_{F} phase. These calculations employed a simulation box containing 384 RM734 molecules with periodic boundary conditions, equilibrated in the NPT ensemble at p=1 atm for a range of temperatures spanning the N and N_{F} phases, using the APPLE&P force field, which has been successfully applied in previous studies of 5CB nematic, and CB7CB twist-bend phases.
Force Field—All molecular dynamics (MD) simulations were conducted using the Atomistic Polarizable Potentials for Liquids, Electrolytes and Polymers (APPLE&P) force field. Parameters for atomic polarizabilities and repulsion-dispersion interactions were taken from the APPLE&P database without modification, while atomic charges were fitted to reproduce the electrostatic field around all of the molecular fragments as obtained from MP²/aug-cc-pVDZ quantum chemistry calculations using Gaussian 16 software. Parameters for missing dihedral potentials were obtained by fitting conformational energy scans obtained from DFT calculations at the M052X/aug-cc-pVDZ level of theory. A non-polarizable version of the force field was also used, with the atomic polarizabilities set to zero and all other parameters kept the same as in the polarizable version.
Simulation Parameters—The simulations were carried out using the WMI-MD simulation package (http://www.wasatchmolecular.com). In these simulations, all covalent bonds were constrained using the SHAKE algorithm. The potential energy of bond angle bending, out-of-plane bending, and dihedral angles was described with harmonic potentials or cosine series expansions. The van der Waals interactions were calculated within a cut-off distance of 12.0 Å, with a smooth tapering to zero starting from 11.5 Å. The charge-charge and charge-induced dipole interactions were calculated using Ewald summation. The induced dipole-induced dipole interactions were truncated at 12.0 Å. To avoid the polarization ‘catastrophe’, a Thole screening parameter of 0.2 was used for small separations between induced dipoles. A multiple time step integration approach was used to enhance computation efficiency. A 0.5 fs time step was used for the calculation of valence interactions, including those involving bonds (SHAKE), bond angle bending, dihedral angles, and out-of-plane deformations. The short-range, non-bonded interactions (with 7.0 Å radius) were calculated every 1.5 fs, while a time step of 3.0 fs was employed for the remaining non-bonded interactions and the reciprocal part of the Ewald summation.
System Initialization and Simulation Protocol—Simulation cells were prepared with two different initial configurations of the molecules: (i) POLAR (POL—all molecules oriented along the +z direction), and (ii) NONPOLAR (NONPOL—equal numbers of molecules oriented along the +z and −z directions). Initially, the 384 molecules were positioned on a relatively low-density lattice with simulation cell dimensions of 150 Å in the x and y directions and 70 Å in the z direction. A 630 ps compression simulation was then conducted to achieve a mass density of ˜1.0 g/cm³ (comparable to typical thermotropic liquid crystal mass densities), with the z-dimension of the simulation cell fixed at 70 Å, and with a biasing potential applied to the ends of the mesogens to preserve their orientation during the initial equilibration stage. The biasing potentials were then removed and further equilibration runs 6 ns in duration and production runs in excess of 20 ns were carried out. All simulations were conducted in the NPT (isobaric, isothermal) ensemble with the z-dimension of the cell fixed and the x and y dimensions allowed to vary to maintain a constant pressure of 1 atm (NPT-XY ensemble). Each system was simulated at 110° C., 130° C., 150° C., and 180° C., temperatures spanning the N_{F}-N phase transition, using polarizable and non-polarizable force fields. The temperature and pressure were controlled with the Nose-Hoover thermostat and barostat.

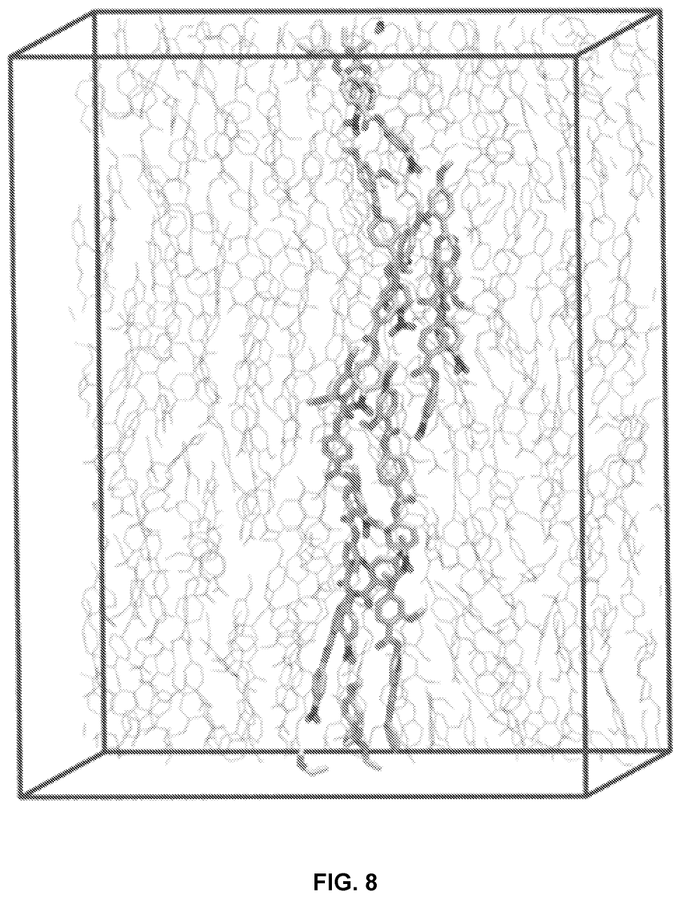

Order parameters—An instantaneous configuration of the POL system at T=130° C. is shown in FIG. 8, revealing that the system retains a high degree of orientational order at this temperature. To quantify nematic orientational order in this system, we measure the traceless, symmetric nematic ordering tensor

$$Q = \frac{1}{N} {\sum }_{α = 1}^{N} ( \frac{3}{2} {u}_{α} {u}_{α} - \frac{1}{2} I ) ,$$

where I is the identity matrix, and where the sum ranges over all molecules. The scalar nematic order parameter S corresponds to the largest eigenvalue of the time-averaged ordering tensor [symbol] Q [symbol] , and the biaxial order parameter B is defined as the difference between the two smallest eigenvalues. Polar order is assessed by measuring the (vector) polar order parameter

$$P = \frac{1}{N} {\sum }_{α = 1}^{N} {u}_{α} ,$$

from which a scalar polar order parameter P= [symbol] P [symbol] can be obtained.

For the POL simulation of the polarizable model at 130° C., we measure a large nematic order parameter, S=0.787±0.009, and a nearly saturated polar order parameter, Π=0.924±0.003, with negligible biaxiality (B=0.013±0.002). Moreover, the polar order parameter P is co-linear with n, the principal eigenvector of [symbol] Q [symbol] . Given that there appear to be no long-range positional correlations (as shown below), the simulated state appears to be a uniaxial ferroelectric nematic (N_{F}) phase.

It is interesting to compare these results with those from the NONPOLAR simulation under the same conditions (T=130° C., polarizable molecular model). The nematic order parameter in this case is S=0.782±0.018, quite similar to that of the polar system, while the polar and biaxial order parameters are small (P=0.013±0.004, B=0.028±0.003), as expected for a conventional uniaxial nematic (N) state. The fact that the magnitude of S is nearly the same in the POL and NONPOL states is generally consistent with the experimental observation that the birefringence does not change significantly through the N-N_{F} transition. Simulated mass density at T=130° C. is δ=1.33 g/cm³.

Ferroelectric polarization density—The measured spontaneous ferroelectric polarization density P in the N_{F} phase of RM734 is large, increasing with decreasing temperature below the N-N_{F} transition to a saturation value of around P=6 μC/cm² (FIG. 3). This implies a high degree of polar order in the N_{F} phase, that can be further quantified by comparison with the polarization P_{S} computed by simulation.

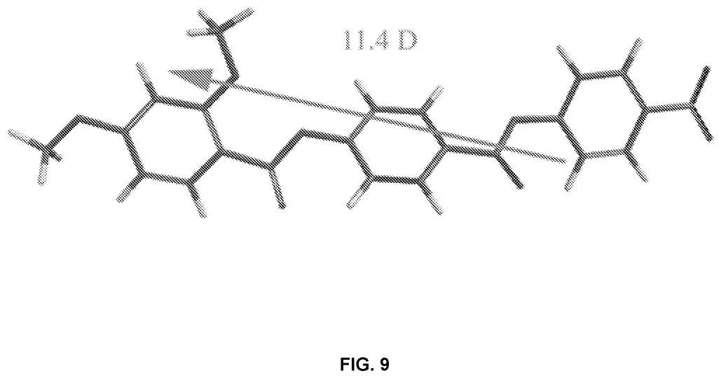

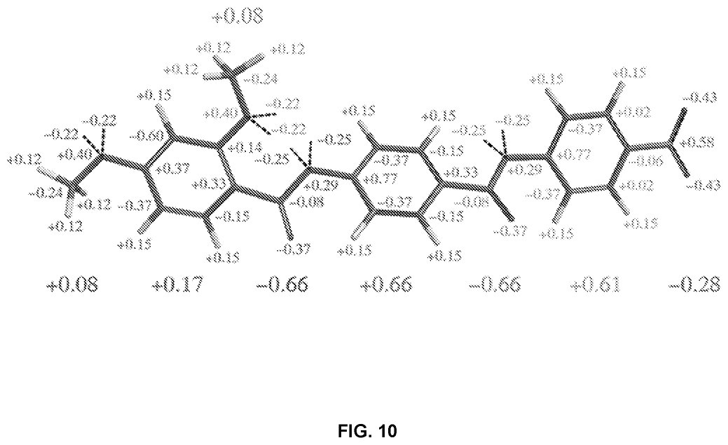

As noted above, RM734 has a large electric dipole moment p=11.4 D, as determined from quantum chemistry calculations at the B3LYP/6-31G* level of theory (FIG. 9). Higher-level quantum chemistry calculations were used to assign site charges to atom sites and lone pair electron sites in the molecular mechanics model used in our atomistic simulations. The resulting static site charges, shown in FIG. 10, are also consistent with a molecular dipole moment of around 11 D (computed using polarization

p=∫rρ(r)dr=Σᵢ₌₁ⁿrᵢqᵢ, where rᵢ and qᵢ are the site positions and charges, and the sum ranges over all n sites in the molecule). Note that the dipole moment has a weak dependence on molecular conformation, and that molecules sample an ensemble of low-energy conformations over the course of a simulation. For the POL simulation of the polarizable model at 130° C., we measure an average static molecular dipole moment (from static site charges) of magnitude [symbol] p_{static} [symbol] =11.24±0.01 D. For the polarizable models, there is also an induced molecular dipole moment component, which has an average magnitude of [symbol] p_{induced} [symbol] =1.46±0.02 D, but has a nearly isotropic orientational distribution, so the average magnitude of the total molecular dipole moment (the sum of static and induced contributions) is nearly equal to the static contribution, [symbol] pₜₒₜₐₗ [symbol] =11.20±0.01 D. The fact that the induced molecular dipole has a nearly isotropic orientational distribution is a consequence of the boundary conditions, which ensure that the average electric field is zero at any point in the system (there is no bound charge at the surface of the system, so the depolarization field vanishes), so the average magnitude of the induced dipole moment vector is close to zero, | [symbol] p_{induced} [symbol] |=0.053±0.009 D.

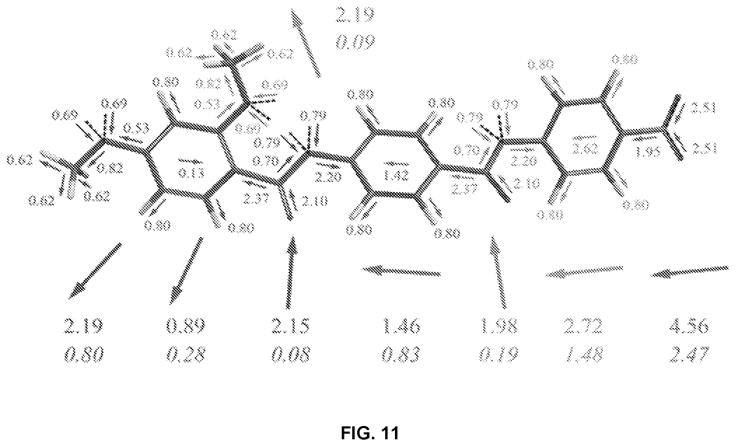

We can gain further insight by resolving the total ferroelectric polarization density into contributions from specific dipolar groups. To accomplish this, we employ a unique decomposition of charges into elementary charge-neutral dipolar groups (bonds and rings), as shown in FIG. 11. Group dipoles can be further aggregated into functional groups, which are or can be indicated by color coding in FIG. 11, together with the dipole moments and average contributions to ferroelectric polarization density P_{S} of specific functional groups. The terminal nitro group and the ring to which it is attached are highly dipolar, and make a dominant (˜64%) contribution to P_{S}. Four functional groups (nitro, nitro ring, central ring, and terminal methoxy) account for ˜90% of the molecular polarization density. We have calculated the average ferroelectric polarization density of the maximally polar equilibrium state of the POL simulation at 130° C. from

$$〈 {P}_{S} 〉 = 〈 \frac{1}{V} {\sum }_{α = 1}^{N} {P}_{α} 〉 ,$$

where V is the system volume. For the POLAR simulation of the polarizable model, the polarization density magnitude P_{S}=| [symbol] P_{S} [symbol] |=6.17±0.01 μC/cm², where only 0.13±0.03 μC/cm² is due to the induced polarization. This calculated P_{S} is in quantitative agreement with the saturation polarization density P measured experimentally (FIG. 3). This value is also nearly the same as that obtained from the simple estimate given in the text assuming 100% polar order, as might be expected given the high value of the POL system polar order parameter given above, Π=0.924. The remanent orientational disorder is due to small orientation fluctuations about n, basically all that is allowed in the POL system. Consequently, Π depends only weakly on T, with the MD giving P_{S}=6.170±0.008 μC/cm² at T=130° C. and P_{S}=6.368±0.002 μC/cm² at T=110° C.

An important inference of this agreement with the N_{F} phase experimental value is that at low T RM734 essentially becomes the POL system, i.e., is a polar nematic with no molecular flips, and remnant polar disorder that is strictly short ranged (˜few molecule) small angle orientation fluctuations about the director. At higher temperatures P decreases in the N_{F} phase because of the growth of longer length scale fluctuations and disordering modes. But these disappear upon cooling to the saturated state at low T, where the fluctuations in the N_{F} become consistent with those allowed in the nanoscale volume and periodic boundary conditions of the POL simulation. On the other hand, the simulation volume is chosen to be large enough to observe the local molecular packing driven by specific intermolecular interactions, discussed next.

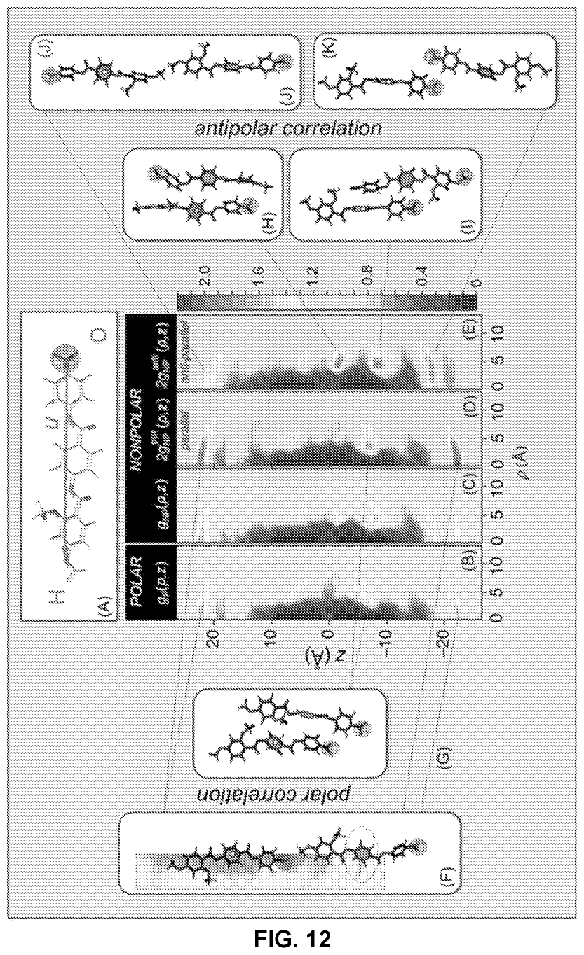

Intermolecular correlations—In order to make headway in understanding the roles of molecular structure and interaction in RM734 and its relation to polar ordering, we applied the atomistic simulations to probe molecular association and packing in the POL and NONPOL systems. We characterized molecular pair positional and orientational correlations by measuring several g(ρ,z), the conditional densities of molecular centers about a molecule fixed with its center at the origin, with its long axis u in FIG. 12(A) parallel to z and pointing in the +z direction [nitro group near (ρ,z)=(0,−10 Å) and the methoxy group near (ρ,z)=(0,10 Å)]. Here the center of the molecule is defined as the midpoint of u in FIG. 12(A), and the g(ρ,z) are angular averages about z of density over the azimuthal orientations of the molecule at the origin. The g(ρ,z) are therefore independent of azimuthal angle φ, reflecting uniaxial nematic symmetry. They all exhibit a correlation hole (g(ρ,z)˜0) around the origin and extended along z for ˜1.5 molecular lengths where other molecular centers are excluded because of steric repulsion and the strong nematic orientational ordering. The pair distributions g_{P}(ρ,z) and g_{NP}(ρ,z) for the POL and NONPOL systems, shown in FIGS. 12(B), and 12(C-E), respectively, display a number of striking features indicating specific molecular association motifs. In the POL system, pronounced arc-shaped peaks observed near (ρ=0 Å, z=+22 Å) indicate a strong tendency for head-to-tail association of parallel pairs of molecules, as illustrated by the representative pair configuration shown in FIG. 12(C). Such head-to-tail configurations are characterized by close association of positively charged H atoms in the terminal methoxy group of the molecule at the origin with negatively charged O atoms in the nitro group of its neighbor, suggesting that head-to-tail association is in large part the result of specific electrostatic interactions. Prominent off-axis peaks near ρ=5 Å, z=±6 Å are also observed in g_{P}(ρ,z), and analysis of pair configurations associated with these peaks (e.g., FIG. 12€) reveals close association of oppositely charged atoms, including close contacts between positively charged H atoms in the lateral methoxy group and the negatively charged O atom in the terminal methoxy group, and between negatively charged O atoms in the nitro and carbonyl groups and positively charged H atoms in the phenyl rings. These observations also point to specific electrostatic interactions stabilizing polar pair configurations. In the POLAR simulation the lateral methoxys appear to be key to establishing the relative positioning of the side-by-side molecular associates that prefer polar ordering. The presence of a region of reduced probability (‘correlation hole’) in g_{P}(ρ,z) near ρ=5 Å, z=0 Å shows that side-by-side configurations of parallel pairs of molecules are relatively unfavorable, presumably due to the excluded volume of their lateral methoxy groups.

The NONPOL system forces both antiparallel and parallel molecular pairs, which give correlation functions, g_{NP}^{anti}(ρ,z) and g_{NP}^{par}(ρ,z), that exhibit very strongly expressed, polarity-dependent molecular recognition. In the on-axis or nearly on-axis peaks of G_{NP}^{anti}(ρ,z) in FIG. 12G we observe that the z→−z symmetry of g_{NP}^{anti}(ρ,z) is the most strongly broken, as expected since HO—OH association will be different from OH—HO association. Thus, there is a prominent arc-shaped peak near ρ=4 Å, z=—18 Å, arising from HO—OH antiparallel lateral association of terminal nitro groups, illustrated by the representative pair configuration in FIG. 12 (G,K). Pair configurations associated with this peak are characterized by close contacts between negatively charged nitro O atoms and positively charged phenyl H atoms adjacent to the nitro group, suggesting that these configurations are electrostatically stabilized. In contrast, the OH—HO associations between terminal methoxy groups (associated with the peak near ρ=0 Å, z=+21 Å) are very weak (FIG. 12(G,J)).

The NONPOL system contribution to g_{NP}^{anti}(ρ,z) from side-by-side antiparallel pairs (FIG. 12(G-I)), shows a prominent peak near p=5 Å, z=−8 Å, associated with pair configurations characterized by close contacts between positively charged H atoms in the lateral methoxy group and negatively charged nitro O atoms. The side-by-side pair configurations associated with the peak near p=15 Å, z=−2 Å similarly involve close contacts between oppositely charged O and H atoms in the ester groups and phenyl rings. These findings provide further support for the hypothesis that electrostatic interactions between specific oppositely charged atoms play a dominant role in stabilizing the characteristic pair configurations observed in our simulations.

The NONPOL system parallel pair correlation function g_{NP}^{par}(ρ,z) in FIG. 12(F) is very similar to the POL system g_{P}(ρ,z) in FIG. 12C, indicating a nanosegregation of the par and anti components, a mixture of OH—OH—OH and HO—HO—HO chains with the OH—HO at their interfaces. Remarkably, the polar features of g_{P}(ρ,z) are not only dominant in g_{NP}^{par}(ρ,z) but even more pronounced than in g_{P}(ρ,z) itself. This suggests that there are certain polar associations in the POL system that can reduce the overall polar order, but that can be replaced by antipolar associations in the NONPOL system that are more favorable for nearby polar order. In any case the simulations show that the polar correlations in FIG. 12 (B,C,F,E), emergent in both POL and NONPOL systems, are the only packing motifs stabilizing polar order and therefore must be the principal drivers stabilizing the N_{F} phase.

The PLUPOLAR Nematic—The POL simulation equilibrates a state in which end-to-end flipping is kinetically arrested and the periodic boundary conditions constrain the allowed wavelengths of orientation fluctuations to λₓ<55 Å and λ_{z}<70 Å. The remnant short ranged fluctuations create the pair correlations exhibited in FIG. 12, which are confined to the volume ρ<10 Å and z<30 Å about the origin, molecular neighbor separation scales, well within the dimensions of the simulation box. These conditions create a PLUPOLAR (plus quam polar) equilibrium state of constrained polar ordering yielding the simulated P_{S} values in FIG. 3 (open circles). Comparing these values with the RM734 data shows that, on the one hand, in the PLUPOLAR state the fluctuations that lead to the phase transition are clearly suppressed, while the remnant short range fluctuations give a P_{S} value exhibiting only a weak dependence on temperature. On the other hand, this P_{S} gives a good account of the polarization density of the N_{F} at low temperature, evidence that at low T the N_{F} phase approaches some comparable PLUPOLAR-like condition of having only short-range fluctuations, and that the simulated g(ρ,z) are characterizing their remnant correlations. This state may be glassy, if the strong T-dependence of the viscosity is any indicator.

The NONPOL system enforces the maximum number of molecular contacts between molecules of opposite orientation. In this situation of maximum polar disorder, possible equilibrated molecular correlations could range from being (i) dominantly antiparallel end-to-end (e.g., OH—HO—OH chains, with side-to-side polar correlations, as in the bilayer smectics of strongly polar molecules); to being (ii) polar end-to-end (a mixture of OH—OH—OH and HO—HO—HO chains with the OH—HO interactions side-by-side). RM734 is distinctly in the latter category, as, remarkably, the principal polar ordering motifs of FIG. 12 (F,G) are even stronger in the NONPOL system than in the POL (compare FIGS. 12(B) and 12(D)), and the antipolar correlations are largely side-by side. The OH—HO end-to-end antipolar association of FIG. 12(J) is present but weak, as is the HO—OH end-to-end pairing of FIG. 12(K). The latter is dominant in the crystal phase, but not as a mode of achieving antipolar ordering in the NONPOL system. It appears from these results that only the polar correlations identified in the POL system (and persisting in the NONPOL system in the maximal presence of enforced polar disorder) could be responsible for the stabilizing the N_{F} phase. Analysis of pair configurations associated with these peaks (e.g., FIG. 1 (F,G)) reveals close association of oppositely charged atoms, including contacts between positively charged H atoms in the lateral methoxy group and the negatively charged O atom in the terminal methoxy group, and between negatively charged O atoms in the nitro and carbonyl groups and positively charged H atoms in the phenyl rings and terminal methyl. These observations suggest that specific electrostatic interactions play a dominant role in stabilizing such pair configurations.

The POL simulation equilibrates a state in which end-to-end flipping is kinetically arrested and the periodic boundary conditions suppress long-wavelength orientation fluctuations (λₓ>55 Å and λ_{z}>70 Å). The remnant short ranged fluctuations create the pair correlations exhibited in FIG. 12, which are confined to the volume ρ<10 Å and z<30 Å about the origin, which are molecular neighbor separation scales well within the dimensions of the simulation box. These conditions create a PLUPOLAR (plus quam polar) equilibrium state of constrained polar ordering yielding the simulated P values in FIG. 3 (open circles). Comparing these values with the RM734 data shows that, on the one hand, in the PLUPOLAR state the fluctuations that lead to the phase transition are clearly suppressed, while the remnant short range fluctuations give a P value exhibiting only a weak dependence on temperature. On the other hand, this P gives a good account of the polarization density of the N_{F} at low temperature, evidence that at low T the N_{F} phase approaches a comparable PLUPOLAR-like condition of having only short-range fluctuations, and that the simulated g(ρ,z) are characterizing their remnant correlations.

Molecular structure—Statistical physical analysis of the stability of the ferroelectric nematic phase shows that two types of intermolecular interactions are required for generating a ferroelectric nematic phase. These are (1) local (nearest neighbor) interactions which favor parallel ordering or neighboring dipoles; and (2) long range dipole-dipole interactions which generate a macroscopic electric field in the direction of the polarization, coupling to the molecular dipoles. The latter affects the nature of the temperature dependence of the polarization in the vicinity of the transition, whereas the former are the desired stabilization forces. It appears that the desired molecular features are (1) a substantial molecular net dipole parallel to the molecular long axis; (2) having this dipole made up from several localized dipole moments distributed along the molecular long axis; (3) minimal flexible tails to enable dipolar charges to interact in a polar fashion, but provide enough flexibility to suppress crystallization; (4) lateral groups to control the relative positions along the director of side-by-side molecules, to promote their polar order. The extremely broad potential palate of synthesizable organic molecules possessing these properties will enable the development of a variety of ferroelectric nematic molecules, if the history of the liquid crystal field is an indicator.
Scope of Disclosure—Although exemplary embodiments of the present disclosure are set forth herein, it should be appreciated that the disclosure is not so limited. For example, although devices are described in connection with specific fluids and molecules, the disclosure is not necessarily limited to these examples. Various modifications, variations, and enhancements of the devices and methods set forth herein may be made without departing from the spirit and scope of the present disclosure.

The subject matter of the present disclosure includes all novel and nonobvious combinations and subcombinations of the various systems, components, and configurations, and other features, functions, acts, and/or properties disclosed herein, as well as any and all equivalents thereof. Further, the claims are hereby incorporated into and form part of the disclosure.

1. A device comprising a volume comprising ferroelectric nematic liquid crystal-forming fluid and means for containing said fluid, said fluid comprising molecules having one or more electric dipoles, said molecules having spontaneously formed a ferroelectric polarization density, said spontaneous polarization density comprising a nonzero local unidirectional average orientation of said dipoles, and said polarization density comprising a magnitude and a vectorial direction in said volume.

2. The device of claim 1 for electrical control of an electromagnetic field, wherein said device includes one or more electrodes for application of an electric field to said volume, and the electromagnetic field propagates in said volume, said electric field causing said polarization density to change in magnitude, thereby producing a change in the electromagnetic field.

3. The device of claim 1 for electrical control of an electromagnetic field, wherein said device includes one or more electrodes for application of an electric field to said volume, and an electromagnetic field to be controlled propagates in said volume, said electric field causing said polarization density to change the vectorial direction, thereby producing a change in the electromagnetic field.

4. The device of claim 1 for producing electrically-driven motion, wherein said device includes one or more electrodes for application of an electric field to said volume, said electric field causing said polarization density to change in the vectorial direction and/or the magnitude, thereby producing a physical motion of or change of shape of said volume.

5. The device of claim 1 for performing mechanical sensing, wherein said device includes one or more electrodes for measuring the electric potential or current flow within said volume, said electric potential and/or current flow generated by change in said polarization density, said change due to a variation in stress within said volume or change of shape of at least a portion of said volume.

6. The device of claim 1 for thermally generating a charge density, wherein said device includes one or more electrodes for measuring an electric potential or obtaining a current flow within said volume, said electric potential and/or current flow generated by a change in said polarization density, said change of said polarization density produced by a change in temperature of said volume.

7. The device of claim 1, wherein said volume is contained between parallel surfaces.

8. The device of claim 7, wherein an electric field is applied parallel to the surfaces.

9. The device of claim 7, wherein the polarization density is parallel to said surfaces.

10. The device of claim 7 wherein said electromagnetic field has a polarization parallel to the surfaces.

11. The device of claim 2, wherein said electric field, said polarization density, and a polarization of said electromagnetic field are along the same line.

12. The device of claim 2, wherein the electromagnetic field comprises one or more of microwave, infrared, visible, ultraviolet, and x-ray light, propagating in or reflecting from said device.

13. The device of claim 1 for performing molecular dipole scavenging, wherein said polarization density produces local molecular scale cavities, said cavities binding molecules having dipoles in said volume.

14. The device of claim 1, where said ferroelectric nematic liquid crystal-forming fluid comprises dimeric, oligomeric, or polymeric material.

15. The device of claim 1, where said ferroelectric nematic liquid crystal-forming fluid comprises elastomeric material.

16. The device of claim 1, where said ferroelectric nematic liquid crystal-forming fluid comprises a glass.

17. The device of claim 1, wherein the molecules comprise features suitable for the stabilization of a ferroelectric nematic phase comprising one or more of (1) a rod shape having a molecular long axis suitable for nematic liquid crystal ordering; (2) a substantial molecular net dipole parallel to the molecular long axis, said dipole stabilizing head-to-tail chaining of said rod-shaped molecules; (3) molecular sub-components along the molecular length giving localized charges of alternating sign distributed along said molecular long axis; (4) minimal flexible tails to enable dipolar charges to interact, but provide enough flexibility to suppress crystallization; and (5) lateral groups to control the relative positions along the director of side-by-side molecules, to promote their polar order.

18. A method of using any of the devices of claim 1.

19. A method for discovering molecular structures with features suitable for stabilization of the ferroelectric nematic phase, said method comprising atomistic molecular dynamic computer simulation, said simulation achieving thermal equilibration of at least two samples of a number of test molecules, said test molecules having a molecular dipolar structure, one of said samples comprising a polar collection of test molecules initiated with maximum polar order of said dipoles, and another one of said samples comprising a nonpolar collection of test molecules initiated with zero polar order of said dipoles, said method comprising the determination and comparison of the mode of forming of polar intermolecular correlations in the polar and nonpolar systems.
